(19) United States
(12) Patent Application Publication
(10) Pub. No.: US 2015/0150779 A1
(43) Pub. Date: Jun. 4, 2015

(54) SINGLE-PHASE TRANSPARENT HAIR GEL

(71) Applicant: Henkel AG & Co. KGaA, Duesseldorf (DE)

(72) Inventors: Jens Delowsky, Norderstedt (DE); Thomas Hippe, Appen (DE)

(21) Appl. No.: 14/614,637

(22) Filed: Feb. 5, 2015

Related U.S. Application Data
(63) Continuation of application No. PCT/EP2013/064217, filed on Jul. 5, 2013.

(30) Foreign Application Priority Data
Aug. 8, 2012 (DE) 10 2012 214 046.8

(51) Int. Cl. A61K 8/88; A61K 8/85; A61K 8/49; A61Q 5/12; A61K 8/73

(52) U.S. Cl. CPC A61K 8/88; A61Q 5/12; A61K 8/73; A61K 8/4946; A61K 8/85

(57) ABSTRACT

The invention relates to single-phase transparent hair-conditioning gels, comprising a cationic polymer, a polyhydroxy compound, and a cationic surfactant and/or an amine in a cosmetically acceptable carrier, to the use of the composition for haircare, and to a method of caring for the hair using the composition and a process for preparing the composition.

### FIELD OF THE INVENTION

The present invention generally relates to single-phase, transparent, hair-conditioning gels containing a cationic polymer, a polyhydroxy compound and a cationic surfactant and/or an amine in a cosmetically acceptable carrier, to the use of the agent for haircare as well as to a method for haircare using the agent and a method of producing the agent.

### BACKGROUND OF THE INVENTION

The significance of care products having the longest-lasting possible effect is increasing, not least as a result of severe stress on the hair e.g. because of coloring or permanent waves, but also as a result of cleaning the hair with shampoos and of environmental stresses.

The reason for this is that the hair can be damaged by treatment/stress, both externally and in its structure, giving it an unattractive appearance which can be perceived by a lack of smoothness and softness, a lack of gloss but also by poorer combability, hair breakage or split ends.

It has therefore been customary for a long time to subject the hair to a special after-treatment to deliver care substances to the hair and scalp which impart an attractive external appearance to the hair again and strengthen the hair structure as well as caring for the scalp or protecting it from drying out. In these after-treatments, the hair is treated with special active agents, for example quaternary ammonium salts or special polymers, generally in the form of a rinse. As a result of this treatment, depending on the formulation, the combability, hold, fullness and gloss of the hair can be improved.

However, the incorporation of skin- and hair-conditioning agents into a cosmetically suitable—usually aqueous—base still continues to present the manufacturer of these compositions with great difficulties even today, since in particular mineral, natural or synthetic fat, wax and oil components, which have been proven to leave behind a caring and conditioning effect on the skin and hair, cannot be incorporated into a cosmetic base by simple mixing.

In particular when silicone oils and/or vegetable fats or oils are incorporated into hair cosmetic compositions as active agents, at least two-phase or multi-phase formulations are generally obtained. This is observed all the more the higher the content of silicone oils and/or vegetable fats and oils in the compositions. These two-phase systems have to be converted to an only briefly stable single-phase system by the consumer before use by vigorous shaking. Even when these products are properly applied, good product performance on the hair does not always exist since, for example, if shaken for too short a time, it is not always possible to ensure even distribution of the active agents. In these cases, it can occur that when the hair-conditioning agent is applied, instead of the required quantity of the active agents, more “carrier substance” ends up on the hair, resulting in a reduced conditioning effect.

At the same time, in the event of incorrect application of this kind, the hair-conditioning agent becomes more concentrated after each application, and from a certain level there can even be over-conditioning which results in the hair becoming heavy.

The object of the present invention was therefore to produce a stable, clear and single-phase hair-conditioning agent, which cares for the hair and increases the combability and gloss of the hair Ideally, the hair-conditioning agents should nevertheless exhibit a relatively low viscosity and, in the event of a higher viscosity, a low yield point so that they can be easily distributed on the hair surface.

It was a further object of the invention to produce a stable, clear and single-phase hair-conditioning agent which allows the incorporation of relatively large quantities of oil components—in particular silicones and/or vegetable oils—without any negative effect on the single-phase nature or the desired viscosity.

A further object of the invention consisted in the fact that not only the collective of hair overall but even the individual hair fibers should appear evenly cared for and glossy from root to tip and have a pleasant tactile feel.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A transparent hair-conditioning agent in gel form, containing a) a cationic polymer, consisting of at least one monomer of formula (I) and optionally a monomer of formula (II)

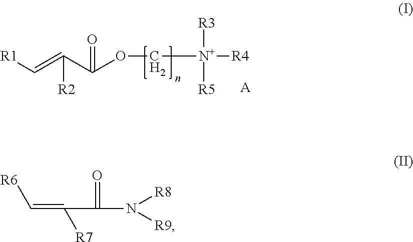

in which R1 to R9 independently of one another signify hydrogen, C₁₋₄ alkyl, methyl, ethyl, propyl, isopropyl, butyl, isobutyl or tert-butyl with the proviso that at least one of the residues R6, R7, R8 or R9 signifies C₁₋₄ alkyl, n denotes integers from 1 to 8 and A denotes a physiologically acceptable anion such as halide, fluoride, chloride, bromide, iodide, hydrogen sulfate or methosulfate; b) at least one cationic compound of formula (Tkat2),

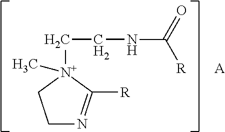

in which the residues R each independently of one another denote a saturated or unsaturated, linear or branched hydrocarbon residue with a chain length of 8 to 30 carbon atoms and A denotes a physiologically acceptable organic or inorganic anion and is selected from the halide ions, fluoride, chloride, bromide, iodide, sulfates of the general formula RSO₃⁻, where R has the meaning of saturated or unsaturated alkyl residues with 1 to 4 carbon atoms, or anionic residues of organic acids such as maleate, fumarate, oxalate, tartrate, citrate, lactate or acetate; c) at least 15.0 to 55.0 wt. % of at least one polyhydroxy compound selected from glycol and/or glycerol and/or 1,2-propylene glycol and/or 1,3-propylene glycol and/or 1,2-pentanediol and/or 1,5-pentanediol and/or 1,2-hexanediol and/or 1,6-hexanediol and/or mixtures thereof; and d) a cosmetic carrier.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has been found that certain cationic polymers in combination with selected polyhydroxy compounds and/or selected cationic surfactants and/or with certain amines achieve the aforementioned aims to a high degree. Their combination enables single-phase products with suitable viscosity, yield point and clarity to be produced.

At the same time, the use of these combinations leads to surprisingly good properties of the treated hair, in particular to improved combability and to improved elasticity. In particular, however, the gloss of the hair treated therewith is increased significantly. Finally, these gels themselves have a novel feel and, even with the smallest quantities of oil components, feel rich, voluminous and oily although they contain only extremely small quantities. These tactile properties clearly differentiate the gels according to the invention from gels of the prior art, particularly as the oils according to the invention have a clear appearance.

The overall composition can be distributed on the hair very evenly. The gel according to the invention, although it has the typical gel consistency, can be distributed very easily and pleasantly on the hair, and even on an individual fiber, using the fingers without applying any force.

The present invention therefore firstly provides a single-phase, transparent hair-conditioning agent in gel form, containing
a) a cationic polymer, consisting of at least one monomer of formula (I) and optionally a monomer of formula (II)

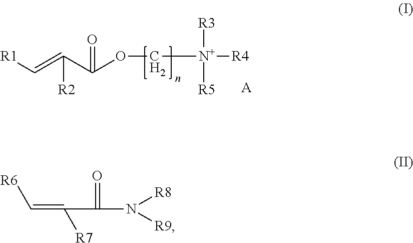

in which R1 to R9 independently of one another signify hydrogen, C₁-C₄ alkyl, methyl, ethyl, propyl, isopropyl, butyl, isobutyl or tert-butyl with the proviso that at least one of the residues R6, R7, R8 or R9 signifies C₁₋₄ alkyl, n denotes integers from 1 to 8 and A denotes a physiologically acceptable anion such as halide, fluoride, chloride, bromide, iodide, hydrogen sulfate or methosulfate,
b) at least one cationic compound of formula (Tkat2),

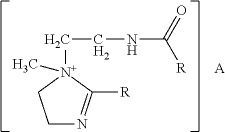

in which the residues R each independently of one another denote a saturated or unsaturated, linear or branched hydrocarbon residue with a chain length of 8 to 30 carbon atoms and A denotes a physiologically acceptable organic or inorganic anion and is selected from the halide ions, fluoride, chloride, bromide, iodide, sulfates of the general formula RSO₃⁻, where R has the meaning of saturated or unsaturated alkyl residues with 1 to 4 carbon atoms, or anionic residues of organic acids such as maleate, fumarate, oxalate, tartrate, citrate, lactate or acetate, and
c) at least 15.0 to 55.0 wt. % of at least one polyhydroxy compound selected from glycol and/or glycerol and/or 1,2-propylene glycol and/or 1,3-propylene glycol and/or 1,2-pentanediol and/or 1,5-pentanediol and/or 1,2-hexanediol and/or 1,6-hexanediol and/or mixtures thereof and
d) a cosmetic carrier.

The ingredients a), b) and c) are described in detail below. Where reference is made below to the active agent complex (A), this statement refers to the ingredients a), b) and c) that are compulsorily contained in the agents according to the invention.

Combability is understood according to the invention to be both the combability of the wet fibers and the combability of the dry fibers. The combing work applied or the force applied during the combing process of a collective of fibers is used as a measure of combability. The measurement parameters can be evaluated by the person skilled in the art by sensory means or can be quantified using measuring instruments.

An important aspect of the hair-conditioning agent according to the invention is that it is present as a gel.

The hair treatment agent according to the invention can exhibit a viscosity of at least 15,000 mPas and no more than 50,000 mPas, preferably of no more than 30,000 mPas and in particular of no more than 25,000 mPas and most preferably of 20,000 mPas (measured with a Brookfield DV-II viscometer, spindle 4 at 20 rpm (20 s) and at 20° C.).

The hair-conditioning agent according to the invention is applied—preferably after cleaning the hair—optionally onto wet or dry hair. The hair is then styled as usual. It may be helpful here to use a hair dryer, hair straightener or at least a comb.

A suitable cosmetic carrier d) for the hair-conditioning agents according to the invention is preferably aqueous and/or aqueous/alcoholic. It preferably contains at least 20 wt. % water. Aqueous-alcoholic cosmetic carriers within the meaning of the present invention are to be understood as aqueous solutions containing 3 to 70 wt. % of a C₁-C₆ alcohol, in particular methanol, ethanol or propanol, isopropanol, butanol, isobutanol, tert.-butanol, n-pentanol, isopentanols, n-hexanol and isohexanols. The agents according to the invention can additionally contain other organic solvents, such as e.g. methoxybutanol, benzyl alcohol or ethyl diglycol. In a more preferred embodiment of the invention, the hair-conditioning agents contain—based on their total weight—at least 20 wt. %, preferably at least 25 wt. %, more preferably at least 30 wt. % and in particular at least 35 wt. % and in the best embodiment according to the invention at least 40 wt. % of an aqueous or aqueous-alcoholic carrier.

As ingredient a), the agents according to the invention contain at least one cationic polymer. The cationic polymer here is made up of at least one monomer of formula (I) and optionally a monomer of formula (II),

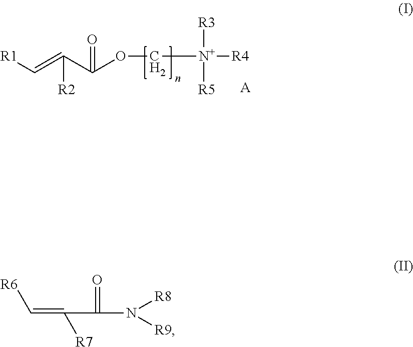

in which R1 to R9 independently of one another signify hydrogen, C1-C4 alkyl, methyl, ethyl, propyl, isopropyl, butyl, isobutyl or tert-butyl with the proviso that at least one of the residues R6, R7, R8 or R9 signifies C1-C4 alkyl, n is an integer from 1 to 8 and A denotes a physiologically acceptable anion, such as halide, fluoride, chloride, bromide, iodide, hydrogen sulfate or methosulfate. R1 is preferably hydrogen or a methyl group, and R1 is more preferably a methyl group. R2 is preferably hydrogen or a C1-C4 alkyl group, and R2 is more preferably hydrogen. R3, R4 and R5, independently of one another, are preferably hydrogen or a C1-C4 alkyl group, in particular a methyl group. More preferably, R3, R4 and R5 are identical and most preferably methyl. The number n is preferably an integer from 1 to 7, more preferably from 1 to 5, extremely preferably 1, 2, 3 or 4 and most preferably 2. R6 is preferably hydrogen or methyl. R7 is preferably hydrogen, methyl or ethyl, more preferably methyl. R8 and R9 are preferably identical and denote hydrogen, methyl or C1-C4 alkyl, more preferably hydrogen or methyl. Most preferred as monomer of formula (II) is dimethyl acrylamide. A cationic polymer according to the invention consists of 20 to 95 wt. % of the monomer (I) and 5 to 50 wt. % of the monomer (II). Preferably, the monomer (I) is contained in an amount of 40 to 90 wt. % and the monomer (II) in an amount of 10 to 40 wt. %. More preferably, the cationic polymer according to the invention from fonnulae (I) and (II) is a homopolymer of monomers of formula (I).

The cationic polymer according to the invention can, of course, also be crosslinked In this case, the crosslinking is effected by the conventional crosslinking agents, such as e.g. allyl acrylamide, allyl methacrylamide, tetraallyl ammonium chloride or N,N′-methylene bisacrylamide in quantities of up to 500 ppm.

According to the invention, the cationic polymer is present as the pure substance in powder form. Other additives, such as solvents, adjusting agents etc., are not contained according to the invention in the cationic polymers used according to the invention. Cationic polymers according to the invention are obtainable e.g. from a monomer of formula (I) and acrylamide. These are e.g. products with the trade names Hercofloc® 812 (Hercules), BINA® Quat P100 (Ciba or BASF), Reten® grades (Hercules), Salcare® SC63 (Ciba or BASF), Rohagit KF 720F (Rohm GmbH, Evonik). Extremely preferred cationic polymers according to the invention are homopolymers of the monomers according to formula (I), such as e.g. the products with the INCI names polyquaternium-9, polyquaternium-14, polyquaternium-45, Quaternium-49 and extremely preferably the commercial product Rheocare® Ultragel (polyquaternium-37).

The cationic polymer according to the invention is used in a quantity of 0.1 to 10 wt. %, based on the total composition. Preferred are quantities of 0.1 to 5 wt. %, more preferably of 0.1 to 3.0 wt. %.

The second essential ingredient is a cationic imidazole derivative. The formula (Tkat2) illustrated below shows the structure of these compounds.

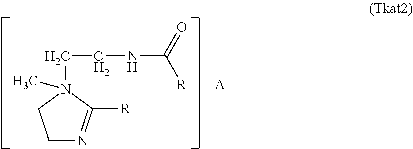

The residues R independently of one another each denote a saturated or unsaturated, linear or branched hydrocarbon residue with a chain length of 8 to 30 carbon atoms. The preferred compounds of the formula (Tkat2) each contain the same hydrocarbon residue for R. The chain length of the residues R is preferably 12 to 21 carbon atoms. A denotes a physiologically acceptable organic or inorganic anion and is selected from the halide ions, fluoride, chloride, bromide, iodide, sulfates of the general formula RSO₃⁻, where R has the meaning of saturated or unsaturated alkyl residues with 1 to 4 carbon atoms, or anionic residues of organic acids such as maleate, fumarate, oxalate, tartrate, citrate, lactate or acetate. Examples that are particularly in accordance with the invention are available e.g. with the INCI names quaternium-27, quaternium-72, quaternium-83 and quaternium-91. Extremely preferred according to the invention are quaternium-27 and quaternium-91.

The cationic surfactant of the formula (Tkat2) according to the invention is used in a quantity of 0.1 to 10 wt. %, based on the total composition. Preferred are quantities of 0.1 to 7.5 wt. %, more preferably 0.2 to 5.0 wt. % and extremely preferably 0.3 to 3.0 wt. %.

The ingredient c) is at least one polyhydroxy compound. This polyhydroxy compound is selected from glycol and/or glycerol and/or 1,2-pentanediol and/or 1,5-pentanediol and/or 1,2-hexanediol and/or 1,6-hexanediol and/or mixtures thereof. The clarity of the compositions according to the invention is produced exclusively by the polyhydroxy compounds. To this end, it is furthermore necessary to use the polyhydroxy compounds in a narrowly limited range of quantities. With smaller or larger quantities, clear compositions are no longer obtained. According to the invention, therefore, at least 15.0 to 55.0 wt. % of at least one polyhydroxy compound selected from glycol and/or glycerol and/or 1,2-propylene glycol and/or 1,3-propylene glycol and/or 1,2-pentanediol and/or 1,5-pentanediol and/or 1,2-hexanediol and/or 1,6-hexanediol and/or mixtures thereof are used in the compositions according to the invention. More preferably, glycol and/or glycerol and/or mixtures thereof are used. Glycol is understood to be 1,2-ethylene glycol. Preferred is a polyhydroxy compound selected from glycol, glycerol, 1,2-propylene glycol and/or 1,3-propylene glycol and mixtures thereof. According to the invention, the best results are obtained if the polyhydroxy compound is selected from glycol and glycerol and mixtures thereof.

The present invention secondly provides a method of producing a cosmetic agent containing a composition according to claim 1. The method according to the invention for producing clear gels from the ingredients a) to d) according to the invention comprises the following process steps, which can be implemented in the order according to the invention. Firstly, the cationic polymer of the invention according to a) is pre-swollen in water at a temperature of 15 to 50° C. The cationic surfactant b) is added to this swelling of the cationic polymer a) with stirring. At this point in time, the composition looks milky and turbid. The pH value of the composition is adjusted to a slightly acidic pH value of about pH 4 to 5 using conventional acids or bases as appropriate. Furthermore at this point, further optional ingredients are added as appropriate. As the last ingredient, the polyhydroxy compound is added with stirring. Finally, the pH value of the composition is again adjusted to the desired value of between 2 and 6.5. Only by adding the polyhydroxy compound c) with stirring is the clarity according to the invention on the one hand and the gel structure and its viscosity according to the invention on the other hand obtained. The viscosity is measured as already described above by the Brookfield method at 25° C. The clarity or transparency is determined visually on the one hand. To this end, writing in Arial font size 10 is placed directly behind a conventional 250 ml beaker containing the composition and is read through said composition. Another way of determining the clarity of the compositions according to the invention consists in determining the turbidity or transparency of the composition according to DIN/EN 27027 (ISO 7027) as a transmitted light turbidity measurement. As the result, the transparency is obtained as a percentage of transmittance. The compositions according to the invention are very slightly milky or opaque to clear. The transparency is between 50 and 100%.

The method according to the invention for producing a cosmetic composition according to claim 1 is characterized as follows:
a) pre-swelling of the cationic polymer according to claim 1 at a temperature of 15 to 50° C.,
b) addition of the cationic surfactant according to claim 1 with further stirring,
c) adjustment of the pH value to 4 to 5 and optional addition of further water-soluble substances,
d) addition of the polyhydroxy compound according to claim 1 with stirring and optional final adjustment of the pH value of the composition to a pH value of 2 to 6.5.

In order to maintain the transparency of the composition according to the invention, the other optional ingredients should also fulfill a condition. These should be able to form a clear solution in water at a concentration of 0.1%.

As the first optional further ingredient, the hair treatment agents according to the invention contain quaternary ammonium compounds in a total quantity of 0.1 to 10 wt. %. Preferred hair treatment agents according to the invention are characterized in that they contain 0.1 to 8.0 wt. %, preferably 0.2 to 8.0 wt. %, more preferably 0.5 to 8.0 wt. %, still more preferably 0.5 to 6 wt. %, still more preferably 1.0 to 6 wt. % and in particular 1.0 to 5.0 wt. % quaternary ammonium compounds.

Quaternary ammonium compounds are, in principle, monomeric cationic or amphoteric ammonium compounds, monomeric amines, aminoamides, polymeric cationic ammonium compounds and polymeric amphoteric ammonium compounds. From this large number of possible quaternary ammonium compounds, the following groups have proved to be particularly suitable and are each individually employed in a quantity of 0.1 to 10.0 wt. %. The quantity used does not fall above or below this range even if a mixture of different compounds of the quaternary ammonium compounds is used.

Esterquats according to the formula (Tkat1-2) form the first group.

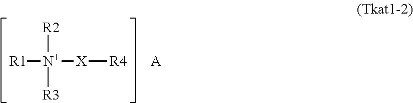

The residues R1, R2 and R3 here are each independent of one another and can be the same or different. The residues R1, R2 and R3 signify:
a branched or unbranched alkyl residue with 1 to 4 carbon atoms, which can contain at least one hydroxyl group, or
a saturated or unsaturated, branched or unbranched or a cyclic saturated or unsaturated alkyl residue with 6 to 30 carbon atoms, which can contain at least one hydroxyl group, or
an aryl or alkaryl residue, e.g. phenyl or benzyl,
the residue (—X—R4), with the proviso that no more than 2 of the residues R1, R2 or R3 can denote this residue.

The residue —(X —R4) is contained at least 1 to 3 times.

X here denotes:
1) —(CH2)n- with n=1 to 20, preferably n=1 to 10 and more preferably n=1-5, or
2) —(CH2-CHR5-O)n- with n=1 to 200, preferably 1 to 100, more preferably 1 to 50 and more preferably 1 to 20 with R5 in the meaning of hydrogen, methyl or ethyl,
3) a hydroxyalkyl group with one to four carbon atoms, which can be branched or unbranched and which contains at least one and no more than 3 hydroxy groups. Examples are: —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues,
   and R4 denotes:
1) R6-O—CO, where R6 is a saturated or unsaturated, branched or unbranched or a cyclic saturated or unsaturated alkyl residue with 6 to 30 carbon atoms, which can contain at least one hydroxy group and which can optionally also be ethoxylated with 1 to 100 ethylene oxide units and/or 1 to 100 propylene oxide units, or
2) R7-CO, where R7 is a saturated or unsaturated, branched or unbranched or a cyclic saturated or unsaturated alkyl residue with 6 to 30 carbon atoms, which can contain at least one hydroxy group, and which can optionally also be ethoxylated with 1 to 100 ethylene oxide units and/or 1 to 100 propylene oxide units,
   and A denotes a physiologically acceptable organic or inorganic anion and is defined at this point in representation of all the structures described, including those below. The anion in all of the cationic compounds described is selected from the halide ions, fluoride, chloride, bromide, iodide, sulfates of the general formula RSO₃⁻, where R has the meaning of saturated or unsaturated alkyl residues with 1 to 4 carbon atoms, or anionic residues of organic acids such as maleate, fumarate, oxalate, tartrate, citrate, lactate or acetate.

Products of this type are marketed e.g. with the trade marks Rewoquat®, Stepantex , Dehyquart®, Armocare® and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90 and Akypoquat® 131 are examples of these esterquats.

Other more preferred compounds according to the invention of the formula (Tkat1-2) are included in the formula (Tkat1-2.1), the cationic betaine esters.

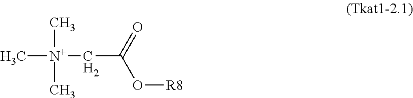

R8 corresponds in its meaning to R7.

More preferred are the esterquats with the trade names Armocare VGH-70, and

Dehyquart F-75, Dehyquart L80, Stepantex VS 90 and Akypoquat® 131.

Cationic surfactants of the formula (Tkat1-1) can likewise be used.

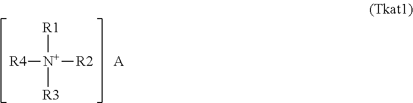

In the formula (Tkat1), R1, R2, R3 and R4 each independently of one another denote hydrogen, a methyl group, a phenyl group, a benzyl group, a saturated, branched or unbranched alkyl residue with a chain length of 8 to 30 carbon atoms, which can optionally be substituted with one or more hydroxy groups. A denotes a physiologically acceptable anion, e.g. halides such as chloride or bromide as well as methosulfates.

Examples of compounds of the formula (Tkat1) are lauryltrimethylammonium chloride, cetyltrimethylammonium chloride, cetyltrimethylammonium bromide, cetyltrimethylammonium metho sulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide, behenyltrimethylammonium methosulfate.

In a more preferred embodiment of the invention, the agents according to the invention also contain at least one amine and/or cationized amine, in particular an amidoamine and/or a cationized amidoamine with the following structural formulae:

R1-NH—(CH₂)ₙ—N⁺R²R³R⁴A (Tkat3)

where R1 signifies an acyl or alkyl residue with 6 to 30 C atoms, which can be branched or unbranched, saturated or unsaturated, and wherein the acyl residue and/or the alkyl residue can contain at least one OH group, and
R2, R3 and R4 each independently of one another signify
1) hydrogen or
2) an alkyl residue with 1 to 4 C atoms, which can be the same or different, saturated or unsaturated, and
3) a branched or unbranched hydroxyalkyl group with one to 4 carbon atoms with at least one and no more than three hydroxy groups, e.g. —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH and hydroxybutyl residues, and
   A signifies an anion as described above and
   n signifies an integer between 1 and 10.

Preferred is a composition in which the amine and/or the quaternized amine according to the general formulae (Tkat3) is an amidoamine and/or a quaternized amidoamine, where R1 signifies a branched or unbranched, saturated or unsaturated acyl residue with 6 to 30 C atoms, which can contain at least one OH group. Preferred here is a fatty acid residue from oils and waxes, in particular from natural oils and waxes. As examples of these, lanolin, beeswax or candellila waxes are suitable.

Also preferred are those amidoamines and/or quaternized amidoamines, in which R2, R3 and/or R4 in the formula (Tkat3) signify a residue according to the general formula CH₂CH₂OR5, where R5 can have the meaning of alkyl residues with 1 to 4 carbon atoms, hydroxyethyl or hydrogen. The preferred value of n in the general formula (Tkat8) is an integer between 2 and 5.

The alkylamidoamines can either be present as such or can be converted to a quaternary compound in the composition by protonation in an appropriately acidic solution. The cationic alkylamidoamines are preferred according to the invention.

Examples of commercial products of this type according to the invention are Witcamine® 100, Incromine® BB, Mackine® 401 and other Mackine® grades, Adogen® S18V, and as permanently cationic aminoamines: Rewoquat® RTM 50, Empigen® CSC, Swanol® Lanoquat DES-50, Rewoquat® UTM 50, Schercoquat® BAS, Lexquat® AMG-BEO or Incroquat® Behenyl HE.

Other quaternary ammonium compounds are ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, behenyltrimethylammonium chloride, distearyldimethylammonium chloride, lauryldimethylammonium chloride, lauryldimethylbenzylammonium chloride and tricetylmethylammonium chloride. The long alkyl chains of the above-mentioned surfactants preferably have 10 to 22 carbon atoms. The above-mentioned cationic surfactants can be used individually or in any combinations with one another, in quantities between 0.01 and 10 wt. %, preferably in quantities of 0.01 to 7.5 wt. % and particularly preferably in quantities of 0.1 to 5.0 wt. %. The best results of all are obtained in this case with quantities of 0.1 to 3.0 wt. %, based in each case on the total composition of the respective agent.

Other quaternary ammonium compounds are cationic and amphoteric polymers. In this case, the cationic polymers mentioned here are not identical with the compulsory cationic polymers according to the invention. Rather, these cationic polymers can be used in addition.

The cationic and/or amphoteric polymers can be homopolymers or copolymers or polymers based on natural polymers, wherein the quaternary nitrogen groups are contained either in the polymer chain or preferably as a substituent on one or more of the monomers. The monomers containing ammonium groups can be copolymerized with non-cationic monomers. Suitable cationic monomers are unsaturated compounds capable of free-radical polymerization, which carry at least one cationic group, in particular ammonium-substituted vinyl monomers, such as e.g. trialkylmethacryloxyalkylammonium, trialkylacryloxyalkylammonium, dialkyldiallylammonium and quaternary vinylammonium monomers with cyclic groups containing cationic nitrogens, such as pyridinium, imidazolium or quaternary pyrrolidones, e.g. alkylvinylimidazolium, alkylvinylpyridinium or alkylvinylpyrrolidone salts. The alkyl groups of these monomers are preferably lower alkyl groups, such as e.g. C1 to C7 alkyl groups, more preferably C1 to C3 alkyl groups.

The monomers containing ammonium groups can be copolymerized with non-cationic monomers. Suitable comonomers are e.g. acrylamide, methacrylamide; alkyl and dialkyl acrylamide, alkyl and dialkyl methacrylamide, alkyl acrylate, alkyl methacrylate, vinyl caprolactone, vinylcaprolactam, vinylpyrrolidone, vinyl esters, e.g. vinyl acetate, vinyl alcohol, propylene glycol or ethylene glycol, the alkyl groups of these monomers preferably being C1 to C7 alkyl groups, more preferably C1 to C3 alkyl groups.

From the large number of these polymers, the following have proved to be particularly effective constituents of the active agent complex according to the invention:

A number of examples of more preferred polymers are described below.

Suitable other cationic polymers—different from a)—can be homopolymers or copolymers, wherein the quaternary nitrogen groups are contained either in the polymer chain or preferably as a substituent on one or more of the monomers. The monomers containing ammonium groups can be copolymerized with non-cationic monomers. Suitable cationic monomers are unsaturated compounds capable of free-radical polymerization, which carry at least one cationic group, in particular ammonium-substituted vinyl monomers, such as e.g. trialkylmethacryloxyalkylammonium, trialkylacryloxyalkylammonium, dialkyldiallylammonium and quaternary vinylammonium monomers with cyclic groups containing cationic nitrogens, such as pyridinium, imidazolium or quaternary pyrrolidones, e.g. alkylvinylimidazolium, alkylvinylpyridinium, or alkylvinylpyrrolidone salts. The alkyl groups of these monomers are preferably lower alkyl groups, such as e.g. C1 to C7 alkyl groups, more preferably C1 to C3 alkyl groups.

Suitable polymers with quaternary amine groups are e.g. the polymers described with the names polyquaternium in the CTFA Cosmetic Ingredient Dictionary, such as methylvinylimidazolium chloride/vinylpyrrolidone copolymer (polyquaternium-16) or quaternized vinylpyrrolidone/dimethylaminoethyl methacrylate copolymer (polyquaternium-11).

Of the cationic polymers that can be contained in the agent according to the invention, for example vinylpyrrolidone/dimethylaminoethyl methacrylate methosulfate copolymer, which is marketed by Gaf Co., USA with the trade names Gafquat® 755 N and Gafquat® 734, is suitable, of which Gafquat® 734 is more preferred. Other cationic polymers are e.g. the copolymer of polyvinylpyrrolidone and imidazolimine methochloride marketed by BASF, Germany with the trade name Luviquat® HM 550, the terpolymer of dimethyldiallylammonium chloride, sodium acrylate and acrylamide marketed by Calgon/USA with the trade name Merquat® Plus 3300 and the vinylpyrrolidone/methacrylamidopropyl trimethylammonium chloride copolymer marketed by ISP with the trade name Gafquat® HS 100.

A suitable cationic polymer which is derived from synthetic polymers is commercially available with the name polyquaternium-74.

Suitable cationic polymers that are derived from natural polymers are cationic derivatives of polysaccharides, e.g. cationic derivatives of cellulose, starch or guar. Also suitable are chitosan and chitosan derivatives. Cationic polysaccharides have the general formula G-O-B-N+RₐR_{b}R_{c}A⁻
G is an anhydroglucose residue, e.g. starch or cellulose anhydroglucose;
B is a divalent linking group, e.g. alkylene, oxyalkylene, polyoxyalkylene or hydroxyalkylene;
Rₐ, R_{b} and R_{c} are, independently of one another, alkyl, aryl, alkylaryl, arylalkyl, alkoxyalkyl or alkoxyaryl each having up to 18 C atoms, wherein the total number of C atoms in Rₐ, R_{b} and R_{c} is preferably no more than 20;
A⁻ is a conventional counter-anion and is preferably chloride.

Cationic, i.e. quaternized, celluloses are commercially available with different degrees of substitution, cationic charge densities, nitrogen contents and molecular weights. For example, polyquaternium-67 is commercially available with the names Polymer® SL or Polymer® SK (Amerchol). With the trade name Mirustyle® CP from Croda, another extremely preferred cellulose is available. This is a trimonium and cocodimonium hydroxyethyl cellulose as a derivatized cellulose with the INCI name polyquaternium-72. Polyquaternium-72 can be used either in solid form or already pre-dissolved in an aqueous solution.

Other cationic celluloses are available with the names Polymer JR® 400 (Amerchol, INCI name polyquaternium-10) and Polymer Quatrisoft® LM-200 (Amerchol, INCI name polyquaternium-24). Other commercial products are the compounds Celquat® H 100 and Celquat® L 200. Finally, with trimonium and cocodimonium hydroxyethyl cellulose, another derivatized cellulose with the INCI name polyquaternium-72 is available from Croda with the trade name Mirustyle® CP. Polyquaternium-72 can be used either in solid form or already pre-dissolved in an aqueous solution. More preferred cationic celluloses are polyquaternium-10, polyquaternium-24, polyquaternium-67 and polyquaternium-72.

Suitable cationic guar derivatives are marketed with the trade name Jaguar and have the INCI name guar hydroxypropyltrimonium chloride. Furthermore, particularly suitable cationic guar derivatives are also marketed by Hercules with the name N-Hance®. Other cationic guar derivatives are sold by Cognis with the name Cosmedia®. A preferred cationic guar derivative is the commercial product AquaCat® from Hercules. This raw material is an already pre-dissolved cationic guar derivative. The cationic guar derivatives are preferred according to the invention.

A suitable chitosan is marketed e.g. by Kyowa Oil & Fat, Japan, with the trade name Flonac®. A preferred chitosan salt is chitosonium pyrrolidone carboxylate, which is marketed e.g. by Amerchol, USA, with the name Kytamer® PC. Other chitosan derivatives are readily available on the market with the trade names Hydagen® CMF, Hydagen® HCMF and Chitolam® NB/101.

Finally, cationic polymers based on sugars can also be used with preference according to the invention.

Compounds of this type are e.g. cationic alkyl oligoglucosides, as shown in the following illustration.

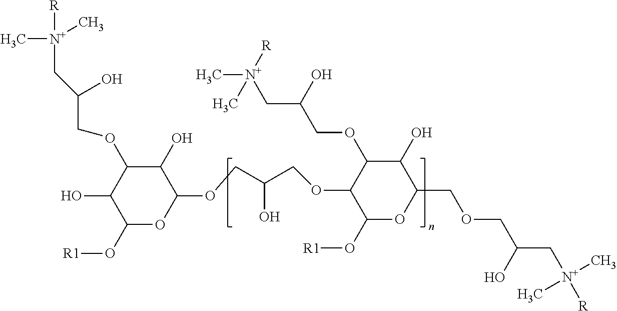

In the formula illustrated above, the residues R independently of one another denote a linear or branched C6 to C30 alkyl residue, a linear or branched C6-C30 alkenyl residue, and the residue R preferably denotes a residue R selected from: lauryl, myristyl, cetyl, stearyl, oleyl, behenyl or arachidyl.

The residues R1 independently of one another denote a linear or branched C6 to C30 alkyl residue, a linear or branched C6 to C30 alkenyl residue; the residue R preferably denotes a residue selected from: butyl, capryl, caprylyl, octyl, nonyl, decanyl, lauryl, myristyl, cetyl, stearyl, oleyl, behenyl or arachidyl. More preferably, the residues R1 are identical. Still more preferably, the residues R1 are selected from technical mixtures of the fatty alcohol blends of C6/C8 fatty alcohols, C8/C10 fatty alcohols, C10/C12 fatty alcohols, C12/C14 fatty alcohols, C12/C18 fatty alcohols, and most preferred here are those technical fatty alcohol blends that are of vegetable origin.

The cationic alkyl oligoglucosides presented above can be prepared e.g. from conventional alkyl oligoglucosides. The alkyl oligoglucosides in this case are reacted by conventional methods to form quaternary ammonium compounds. The alkyl or alkenyl oligoglycosides can be known nonionic surfactants. These sugar surfactants represent known nonionic surfactants according to formula (I),

R¹O-[G]ₚ (I)

in which R¹ denotes an alkyl or alkenyl residue with 4 to 22 carbon atoms, G denotes a sugar residue with 5 or 6 carbon atoms and p denotes numbers from 1 to 10.

The alkyl and alkenyl oligoglycosides can be derived from aldoses or ketoses with 5 or 6 carbon atoms, preferably from glucose. The preferred alkyl and/or alkenyl oligoglycosides are therefore alkyl and/or alkenyl oligoglucosides. The index p in the general formula (I) indicates the degree of oligomerization (DP), i.e. the distribution of mono- and oligoglycosides, and denotes a number between 1 and 30. While p in the individual molecule must always be an integer and can primarily assume the values p=1 to 20 here, the value p for a specific alkyl oligoglycoside is an analytically determined mathematical value, which usually represents a fractional number. Alkyl and/or alkenyl oligoglycosides having an average degree of oligomerization p of 1.1 to 20.0 are usually employed. The alkyl or alkenyl residue R¹ can be derived from primary alcohols with 4 to 30, preferably 6 to 24 carbon atoms, more preferably 8 to 22 carbon atoms. Typical examples are butanol, caproyl alcohol, capryl alcohol, capric alcohol, octanol, nonanol, decanol, undecyl alcohol, lauryl alcohol, myristyl alcohol, cetyl alcohol, oleyl alcohol, stearyl alcohol, behenyl alcohol, arachidyl alcohol and technical mixtures of and with these alcohols.

The quaternization of the alkyl oligoglucosides can be carried out e.g. with quaternary ammonium salts, such as e.g. N,N-dimethyl-N-(n-alkyl)-N-(2-hydroxy-3-chloro-n-propyl)ammonium halides. The chain length of the alkyl group is preferably 6 to 30, more preferably 8 to 24 carbon atoms.

More preferred examples of the cationic alkyl oligoglucosides are the compounds with the INCI names polyquaternium-77, polyquaternium-78, polyquaternium-79, polyquaternium-80, polyquaternium-81 and polyquaternium-82. Most preferred are the cationic alkyl oligogluco sides with the names polyquaternium-77, polyquaternium-81 and polyquaternium-82.

Compounds of this type can be purchased e.g. with the name Poly Suga® Quat from Colonial Chemical Inc.

The cationic alkyl oligoglucosides are used in a total quantity of 0.01 to 10.0 wt. %, preferably 0.05 to 5.0 wt. %, still more preferably 0.1 to 3.0 wt. % and most preferably in quantities of 0.2 to 2.0 wt. %, based in each case on the total weight of the composition. It is, of course, also included in the invention that mixtures of cationic alkyl oligoglucosides can be used. It is preferred in this case if both a long-chain and a short-chain cationic alkyl oligoglucoside are used simultaneously.

Another cationic polymer can be obtained based on ethanolamine. The polymer is commercially available with the name polyquaternium-71.

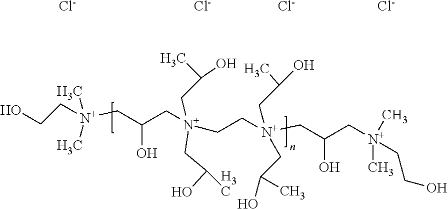

This polymer can be purchased e.g. with the name Cola Moist 300 P from Colonial Chemical Inc.

Polyquaternium-71 is used in a total quantity of 0.01 to 10.0 wt. %, preferably 0.05 to 5.0 wt. %, still more preferably 0.1 to 3.0 wt. % and most preferably in quantities of 0.2 to 2.0 wt. % based in each case on the total weight of the composition.

Other preferred cationic polymers are e.g.
cationized honey, e.g. the commercial product Honeyquat® 50,
polymeric dimethyldiallylammonium salts and their copolymers with esters and amides of acrylic acid and methacrylic acid. The products that are commercially available with the names Merquat® 100 (poly(dimethyldiallylammonium chloride)) and Merquat® 550 (dimethyldiallylammonium chloride-acrylamide copolymer) are examples of these cationic polymers with the INCI name polyquaternium-7,
vinylpyrrolidone-vinylimidazolium methochloride copolymers, as sold with the names Luviquat® FC 370, FC 550 and the INCI name polyquaternium-16 as well as FC 905 and HM 552,
quaternized vinylpyrrolidone/dimethylaminoethyl methacrylate, for example vinylpyrrolidone/dimethylaminoethyl methacrylate methosulfate copolymer, which is sold by Gaf Co., USA, with the trade names Gafquat® 755 N and Gafquat® 734 and the INCI name polyquaternium-11,
quaternized polyvinyl alcohol,
as well as the polymers known by the names polyquaternium-2, polyquaternium-17, polyquaternium-18 and polyquaternium-27 with quaternary nitrogen atoms in the polymer main chain,
vinylpyrrolidone-vinylcaprolactam-acrylate terpolymers, as offered for sale with acrylic acid esters and acrylic acid amides as the third monomer building block, e.g. with the name Aquaflex® SF 40.

Amphoteric polymers according to the invention are those polymers in which a cationic group is derived from at least one of the following monomers:
(i) monomers with quaternary ammonium groups of the general formula (Mono1),

R¹—CH═CR²—CO—Z—(CₙH2ₙ)-N⁽⁺⁾R²R³R⁴A⁽⁻⁾ (Mono 1)
in which R¹ and R², independently of one another, denote hydrogen or a methyl group and R³, R⁴ and R⁵, independently of one another, denote alkyl groups with 1 to 4 carbon atoms, Z is an NH group or an oxygen atom, n is an integer from 2 to 5 and A⁽⁻⁾ is the anion of an organic or inorganic acid,
(ii) monomers with quaternary ammonium groups of the general formula (Mono2),

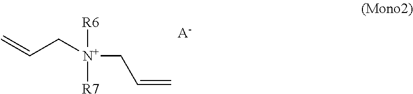

where R⁶ and R⁷, independently of one another, denote a (C₁ to C₄) alkyl group, in particular a methyl group, and
A⁻ is the anion of an organic or inorganic acid,
(iii) monomeric carboxylic acids of the general formula (Mono3),

R⁸—CH═CR⁹—COOH (Mono3)
in which R⁸ and R⁹, independently of one another, are hydrogen or methyl groups.

More preferred are those polymers in which monomers of type (i) are employed, in which R³, R⁴ and R⁵ are methyl groups, Z is an NH group and A⁽⁻⁾ is a halide, methoxysulfate or ethoxysulfate ion; acrylamidopropyltrimethylammonium chloride is a more preferred monomer (i). As monomer (ii) for the above polymers, acrylic acid is preferably used.

More preferred amphoteric polymers are copolymers of at least one monomer (Mono1) or (Mono2) with the monomer (Mono3), in particular copolymers of the monomers (Mono2) and (Mono3). Particularly preferably used amphoteric polymers according to the invention are copolymers of diallyldimethylammonium chloride and acrylic acid. These copolymers are marketed with the INCI name polyquaternium-22, inter alia with the trade name Merquat® 280 (Nalco).

In addition, besides a monomer (Mono1) or (Mono2) and a monomer (Mono3), the amphoteric polymers according to the invention can additionally contain a monomer (Mono4)
(iv) monomeric carboxylic acid amides of the general formula (Mono4),

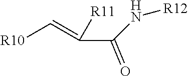

in which R¹⁰ and R¹¹, independently of one another, are hydrogen or methyl groups and R¹² denotes a hydrogen atom or a (C₁ to C₈) alkyl group.

Particularly preferably used amphoteric polymers according to the invention based on a comonomer (Mono4) are terpolymers of diallyldimethylammonium chloride, acrylamide and acrylic acid. These copolymers are marketed with the INCI name polyquaternium-39, inter alia with the trade name Merquat® Plus 3330 (Nalco).

The amphoteric polymers can generally be used according to the invention either directly or in the form of a salt, which is obtained by neutralization of the polymers, for example with an alkali hydroxide.

The above-mentioned cationic polymers can be used individually or in any combinations with one another, with quantities of between 0.01 to 10 wt. %, preferably in quantities of 0.01 to 7.5 wt. % and particularly preferably in quantities of 0.1 to 5.0 wt. % being contained. The best results of all are obtained here with quantities of 0.1 to 3.0 wt. %, based in each case on the total composition of the respective agent.

In another preferred embodiment of the invention, to enhance the haircare further, the hair-conditioning agents additionally contain 0.05 to 15 wt. %, preferably 0.1 to 12 wt. % and in particular 0.25 to 10 wt. % of at least one oil, wax and/or fat component. This/these provide(s) the hair with improved combability and act(s) as (an) extremely effective refatting agent(s).

Suitable oil, wax and/or fat components according to the invention are selected from natural and synthetic oil components and/or fatty substances.

As natural (vegetable) oils, triglycerides and mixtures of triglycerides are generally employed. Preferred natural oils within the meaning of the invention are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, cocoa butter and shea butter. Particularly preferred are almond oil, apricot kernel oil, argan oil, olive oil, jojoba oil, cocoa butter and shea butter. The vegetable oils can be used in the hair-conditioning agents according to the invention either individually or as a mixture of multiple oils.

In particular, mineral oils, paraffin and isoparaffin oils and synthetic hydrocarbons are employed as mineral oils. A hydrocarbon that can be employed according to the invention is e.g. 1,3-di-(2-ethylhexyl)cyclohexane (Cetiol® S), which is available as a commercial product.

Silicone compounds are furthermore suitable as synthetic oils.

Silicones produce excellent conditioning properties on the hair. In particular, they produce better combability of the hair in the wet and dry state and in many cases have a positive effect on hair feel and on the softness of hair.

Suitable silicones according to the invention are selected from among:
(i) polyalkyl siloxanes, polyaryl siloxanes, polyalkylaryl siloxanes, which are volatile or non-volatile, straight-chained, branched or cyclic, crosslinked or non-crosslinked;
(ii) polysiloxanes, which contain in their general structure one or more organofunctional groups that are selected from among:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxy groups;
   l) sulfonic acid groups; and
   m) sulfate or thiosulfate groups;
(iii) linear polysiloxane(A)-polyoxyalkylene(B) block copolymers of the (A-B)ₙ type with n>3;
(iv) grafted silicone polymers with a non-silicone-containing, organic backbone, which consist of an organic main chain which is formed from organic monomers containing no silicone, onto which at least one polysiloxane macromer has been grafted in the chain and optionally on at least one end of the chain;
(v) grafted silicone polymers with a polysiloxane backbone, onto which non-silicone-containing, organic monomers have been grafted, having a polysiloxane main chain onto which at least one organic macromer which contains no silicone has been grafted in the chain and optionally on at least one end thereof;
(vi) or mixtures thereof.

A dialkyl ether can furthermore be used as an oil component.

Dialkyl ethers that can be employed according to the invention are in particular di-n-alkyl ethers with a total of between 12 and 36 C atoms, in particular 12 to 24 C atoms, such as e.g. di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert.-butyl ether, di-isopentyl ether, di-3-ethyldecyl ether, tert.-butyl-n-octyl ether, isopentyl-n-octyl ether and 2-methylpentyl-n-octyl ether.

More preferred according to the invention is di-n-octyl ether, which is commercially available with the name Cetiol® OE.

In another preferred embodiment of the invention, the action of the active agent combination according to the invention can be further optimized by additional fatty substances. Additional fatty substances are to be understood as fatty acids, fatty alcohols and natural and synthetic waxes, which can be present either in solid form or as a liquid in an aqueous dispersion.

As fatty acids, it is possible to use linear and/or branched, saturated and/or unsaturated fatty acids with 6-30 carbon atoms. Preferred are fatty acids with 10-22 carbon atoms. Among these, e.g. the isostearic acids, such as the commercial products Emersol® 871 and Emersol® 875, and isopalmitic acids, such as the commercial product Edenor® IP 95, and all other fatty acids marketed under the trade names Edenor® (Cognis) should be mentioned. Other typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, which are obtained e.g. by hydrolysis of natural fats and oils under pressure, oxidation of aldehydes from Roelen's oxo synthesis or dimerization of unsaturated fatty acids. More preferred generally are the fatty acid blends that are obtainable from coconut oil or palm oil; the use of stearic acid is usually particularly preferred.

The quantity employed in this case is 0.1-15 wt. %, based on the total agent. In a preferred embodiment, the quantity is 0.5-10 wt. %, with quantities of 1-5 wt. % being particularly advantageous.

As fatty alcohols, it is possible to employ saturated, mono- or polyunsaturated, branched or unbranched fatty alcohols with C₆-C₃₀, preferably C₁₀-C₂₂ and particularly preferably C₁₂-C₂₂ carbon atoms. For example, decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol and the Guerbet alcohols thereof can be employed within the meaning of the invention, this list being intended to be of an exemplary and non-limiting nature. However, the fatty alcohols are derived from preferably natural fatty acids, wherein it can generally be assumed that they are obtained from the esters of the fatty acids by reduction. According to the invention, it is likewise possible to employ those fatty alcohol blends that are produced by reduction of naturally occurring triglycerides, such as beef tallow, palm oil, ground nut oil, rapeseed oil, cottonseed oil, soybean oil, sunflower oil and linseed oil or fatty acid esters formed from the transesterification products thereof with corresponding alcohols, and which thus represent a mixture of different fatty alcohols. Substances of this type can be purchased e.g. with the names Stenol®, e.g. Stenol® 1618, or Lanette®, e.g. Lanette®O, or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible according to the invention to employ wool wax alcohols, as can be purchased e.g. with the names Corona®, White Swan®, Coronet® or Fluilan®.

The fatty alcohols are employed in quantities of 0.1-5 wt. %, based on the total preparation, preferably in quantities of 0.1-3 wt. %.

As natural or synthetic waxes, it is possible according to the invention to employ solid paraffins or isoparaffins, carnauba waxes, beeswaxes, candelilla waxes, ozokerites, ceresin, cetaceum, sunflower wax, fruit waxes, such as e.g. apple wax or citrus wax, micro waxes comprising PE or PP. Waxes of this type are available e.g. through Kahl & Co., Trittau.

Other fatty substances are e.g.
ester oils. Ester oils are to be understood as the esters of C₆-C₃₀ fatty acids with C₂-C₃₀ fatty alcohols. Preferred are the monoesters of fatty acids with alcohols having 2 to 24 C atoms. Examples of fatty acid portions that are employed in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, which are obtained e.g. by hydrolysis of natural fats and oils under pressure, oxidation of aldehydes from Roelen's oxo synthesis or dimerization of unsaturated fatty acids. Examples of the fatty alcohol portions in the ester oils are isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linoleyl alcohol, linolenyl alcohol, elaeostearyl alcohol, arachyl alcohol, gadoleyl alcohol, behenyl alcohol, erucyl alcohol and brassidyl alcohol and technical mixtures thereof, which are obtained e.g. by high-pressure hydrogenation of technical methyl esters based on fats and oils or aldehydes from Roelen's oxo synthesis and as a monomer fraction in the dimerization of unsaturated fatty alcohols. More preferred according to the invention are isopropyl myristate (Rilanit® IPM), isononanoic acid C₁₆-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN) and oleic acid decyl ester (Cetiol® V).
dicarboxylic acid esters, such as di-n-butyl adipate, di-(2-ethylhexyl)adipate, di-(2-ethylhexyl)succinate and diisotridecyl acetate as well as diol esters, such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,
symmetric, asymmetric or cyclic esters of carbonic acid with fatty alcohols, glycerol carbonate or dicaprylyl carbonate (Cetiol® CC),
ethoxylated or non-ethoxylated mono-, di- and trifatty acid esters of saturated and/or unsaturated, linear and/or branched fatty acids with glycerol, such as e.g. Monomuls® 90-018, Monomuls® 90-L12, Cetiol® HE or Cutina® MD.

The quantity of the other fatty substances employed is 0.1-20 wt. % based on the total agent. Preferred are 0.1-10 wt. % and more preferably 0.1-5 wt. %, based on the total agent.

More preferred in terms of optimum hair gloss and excellent combability are hair-conditioning agents according to the invention which contain as a further hair-conditioning component d) at least one silicone oil—preferably a dimethicone—and/or at least one vegetable oil. These oils can be incorporated into the hair-conditioning agents according to the invention individually or as a mixture in a quantity of up to 15 wt. % without any negative effect on the single-phase nature and/or the stability of the agents. Furthermore, the required viscosity of the agents for a spray application is achieved despite the high content of the aforementioned preferred hair conditioning oils.

Vitamins are essential to the health of skin and hair. Since they also display a favorable effect when applied externally, they are often added to cosmetic preparations. It has been found that the polymer a) according to the invention in the hair-conditioning agents, besides depositing the components b) and d) on the hair, can also stimulate the deposition of other care substances—including, for instance, vitamins.

Preferred vitamins, provitamins and vitamin precursors and derivatives thereof according to the invention are to be understood as those representatives that are usually allocated to the groups A, B, C, E, F and H.

The group of substances referred to as vitamin A includes retinol (vitamin A₁) and 3,4-didehydroretinol (vitamin A₂). β-Carotene is the provitamin of retinol. Suitable according to the invention as vitamin A component are e.g. vitamin A acid and esters thereof, vitamin A aldehyde and vitamin A alcohol and esters thereof, such as the palmitate and the acetate. The agents according to the invention contain the vitamin A component preferably in quantities of 0.05-1 wt. %, based on the total preparation.

The vitamin B group or vitamin B complex includes, inter alia:
Vitamin B1 (thiamin)
Vitamin B2 (riboflavin)
Vitamin B3. This name often includes the compounds nicotinic acid and nicotinamide (niacinamide). Nicotinamide, which is contained in the agents used according to the invention preferably in quantities of 0.05 to 1 wt. %, based on the total agent, is preferred according to the invention.
Vitamin B₅ (pantothenic acid, panthenol and pantolactone). Within the framework of this group, panthenol and/or pantolactone is preferably employed. Derivatives of panthenol that can be employed according to the invention are, in particular, the esters and ethers of panthenol as well as cationically derivatized panthenols. Individual representatives are, for example, panthenol triacetate, panthenol monoethyl ether and the monoacetate thereof as well as cationic panthenol derivatives. The above compounds of the vitamin B₅ type are preferably contained in the agents according to the invention in quantities of 0.05-10 wt. %, based on the total agent. Quantities of 0.1-5 wt. % are more preferred.
Vitamin B₆ (pyridoxine as well as pyridoxamine and pyridoxal).

Vitamin C (ascorbic acid). Vitamin C is employed in the agents according to the invention preferably in quantities of 0.1 to 3 wt. %, based on the total agent. Use in the form of the palmitic acid ester, the glucosides or phosphates may be preferred. Use in combination with tocopherols may likewise be preferred.

Vitamin E (tocopherols, in particular α-tocopherol). Tocopherol and its derivatives, including in particular the esters, such as the acetate, nicotinate, phosphate and succinate, are contained in the agents according to the invention preferably in quantities of 0.05-1 wt. %, based on the total agent.

Vitamin F. The term “vitamin F” is usually understood to mean essential fatty acids, in particular linoleic acid, linolenic acid and arachidonic acid.

Vitamin H. The compound (3aS,4S,6aR)-2-oxohexahydrothienol[3,4-d]-imidazole-4-valeric acid is referred to as vitamin H, but its trivial name biotin has now become accepted. Biotin is contained in the agents according to the invention preferably in quantities of 0.0001 to 1.0 wt. %, in particular in quantities of 0.001 to 0.01 wt. %.

The compositions according to the invention preferably contain vitamins, provitamins and vitamin precursors from the groups A, B, E and H. Panthenol, pantolactone, pyridoxine and derivatives thereof, as well as nicotinamide and biotin, are more preferred and are added to the hair-conditioning agents according to the invention both individually and in combination with one another.

Protein hydrolyzates and/or derivatives thereof are another optional but preferred active agent in the compositions according to the invention with the active agent complex according to the invention, as they support the strengthening of the hair structure and prevent the hair from drying out.

According to the invention, protein hydrolyzates of vegetable as well as animal or marine or synthetic origin can be used.

Animal protein hydrolyzates are e.g. elastin, collagen, keratin, silk and milk protein hydrolyzates, which can also be present in the form of salts. Products of this type are marketed e.g. with the trademarks Dehylan® (Cognis), Promois® (Interorgana), Collapuron® (Cognis), Nutrilan® (Cognis), Gelita-Sol (Deutsche Gelatine Fabriken Stoess & Co), Lexein® (Inolex) and Kerasol® (Croda).

Furthermore, preferred vegetable protein hydrolyzates according to the invention are e.g. soybean, almond, pea, potato and wheat protein hydrolyzates. Products of this type are available e.g. with the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Other preferred protein hydrolyzates according to the invention are of marine origin. These include e.g. collagen hydrolyzates from fish or algae as well as protein hydrolyzates from bivalves or pearl hydrolyzates. Examples of pearl extracts according to the invention are the commercial products Pearl Protein Extract BG® or Crodarom® Pearl.

More preferably, keratin, silk, milk, wheat and/or soy protein hydrolyzates are employed in the hair-conditioning agents according to the invention. Particularly preferred are keratin and/or wheat protein hydrolyzates.

The protein hydrolyzates are contained in the compositions—based on their total weight—in quantities of 0.001 wt. % to 20 wt. %, preferably of 0.05 wt. % to 15 wt. % and particularly preferably in quantities of 0.05 wt. % to 5 wt. %.

An optional but preferred further component for stabilizing the hair-conditioning agents according to the invention is a nonionic surfactant and/or a nonionic emulsifier. Nonionic surfactants and/or nonionic emulsifiers are added to the hair-conditioning agents according to the invention—based on their weight—individually or in combination with one another in quantities of 0.01 to 10 wt. %, preferably of 0.05 to 7.5 wt. % and in particular in quantities of 0.1 to 5 wt. %.

Suitable nonionic surfactants according to the invention contain as a hydrophilic group e.g. a polyol group, a polyalkylene glycol ether group or a combination of a polyol group and a polyglycol ether group. Compounds of this type are e.g.
addition products of 2 to 50 moles ethylene oxide and/or 0 to 5 moles propylene oxide to linear and branched fatty alcohols with 8 to 30 C atoms, to fatty acids with 8 to 30 C atoms and to alkylphenols with 8 to 15 C atoms in the alkyl group,
addition products of 2 to 50 moles ethylene oxide and/or 0 to 5 moles propylene oxide to linear and branched fatty alcohols with 8 to 30 C atoms, to fatty acids with 8 to 30 C atoms and to alkylphenols with 8 to 15 C atoms in the alkyl group, end-capped with a methyl or C₂-C₆ alkyl residue, such as e.g. the grades that are available with the trade names Dehydol® LS and Dehydol® LT (Cognis),
C₁₂-C₃₀ fatty acid mono- and diesters of addition products of 1 to 30 moles ethylene oxide to glycerol,
addition products of 5 to 60 moles ethylene oxide to castor oil and hydrogenated castor oil, e.g. castor oil hydrogenated +40 EO, as is commercially available e.g. from SHC with the trade name Cremophor CO 455,
polyol fatty acid esters, such as e.g. the commercial product Hydagen® HSP (Cognis) or Sovermol grades (Cognis),
alkoxylated triglycerides,
alkoxylated fatty acid alkyl esters of formula (V)

R¹⁴CO(OCH₂CHR15_{w}OR16 (V)
in which R¹⁴CO denotes a linear or branched, saturated and/or unsaturated acyl residue with 6 to 22 carbon atoms, R¹⁵ denotes hydrogen or methyl, R¹⁶ denotes linear or branched alkyl residues with 1 to 4 carbon atoms and w denotes numbers from 1 to 20,
amine oxides,
hydroxy mixed ethers,
sorbitan fatty acid esters and addition products of ethylene oxide to sorbitan fatty acid esters, such as e.g. the polysorbates,
sugar fatty acid esters and addition products of ethylene oxide to sugar fatty acid esters,
addition products of ethylene oxide to fatty acid alkanolamides and fatty amines,
fatty acid N-alkyl glucamides,
alkyl polyglycosides corresponding to the general formula RO—(Z)ₓ wherein R denotes alkyl, Z denotes sugar and x denotes the number of sugar units. The alkyl polyglycosides that can be used according to the invention can only contain a specific alkyl residue R. However, these compounds are usually produced starting from natural fats and oils or mineral oils. In this case, mixtures corresponding to the starting compounds or corresponding to the respective work-up of these compounds are present as alkyl residues R. More preferred are those alkyl polyglycosides in which R consists
   substantially of C₈ and C₁₀ alkyl groups,
   substantially of C₁₂ and C₁₄ alkyl groups,
   substantially of C₈ to C₁₆ alkyl groups or
   substantially of C₁₂ to C₁₆ alkyl groups or
   substantially of C₁₆ to C₁₈ alkyl groups.

As sugar building block Z, any mono- or oligosaccharides can be employed. Sugars with 5 or 6 carbon atoms and the corresponding oligosaccharides are generally employed. These sugars are e.g. glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is more preferred.

The alkyl polyglycosides that can be used according to the invention contain on average 1.1 to 5 sugar units. Alkyl polyglycosides with x values of 1.1 to 2.0 are preferred. Particularly preferred are alkyl glycosides in which x is 1.1 to 1.8.

The alkoxylated homologs of the above alkyl polyglycosides can also be employed according to the invention. These homologs can contain on average up to 10 ethylene oxide and/or propylene oxide units per alkyl glycoside unit.

In the surfactants which represent addition products of ethylene and/or propylene oxide to fatty alcohols or derivatives of these addition products, both products with a “normal” homolog distribution and those with a narrow homolog distribution can be used. A “normal” homolog distribution here is understood to be mixtures of homologs obtained during the reaction of fatty alcohol and alkylene oxide using alkali metals, alkali metal hydroxides or alkali metal alcoholates as catalysts. Narrow homolog distributions, on the other hand, are obtained when e.g. hydrotalcite, alkaline earth metal salts of ether carboxylic acids, alkaline earth metal oxides, hydroxides or alcoholates are used as catalysts. The use of products with a narrow homolog distribution may be preferred.

More preferred nonionic surfactants are alkyl polyglucosides and alkylene oxide addition products to saturated linear fatty alcohols and fatty acids with in each case 2 to 30 moles ethylene oxide per mole of fatty alcohol or fatty acid. Preparations with excellent mild properties are also obtained if they contain fatty acid esters of ethoxylated glycerol as nonionic surfactants.

Emulsifiers that can be used according to the invention are e.g.
addition products of 4 to 30 moles ethylene oxide and/or 0 to 5 moles propylene oxide to linear fatty alcohols with 8 to 22 C atoms, to fatty acids with 12 to 22 C atoms and to alkylphenols with 8 to 15 C atoms in the alkyl group,
C₁₂-C₂₂ fatty acid mono- and diesters of addition products of 1 to 30 moles ethylene oxide to polyols with 3 to 6 carbon atoms, in particular to glycerol,
ethylene oxide and polyglycerol addition products to methyl glucoside fatty acid esters, fatty acid alkanolamides and fatty acid glucamides,
C₈-C₂₂ alkyl mono- and oligoglycosides and ethoxylated analogs thereof, wherein degrees of oligomerization of 1.1 to 5, in particular 1.2 to 2.0, and glucose as sugar component are preferred,
mixtures of alkyl(oligo)glucosides and fatty alcohols, e.g. the commercially available product Montanov® 68,
addition products of 5 to 60 moles ethylene oxide to castor oil and hydrogenated castor oil,
partial esters of polyols having 3-6 carbon atoms with saturated fatty acids having 8 to 22 C atoms,
sterols. Sterols are understood to be a group of steroids which carry a hydroxyl group on the C atom 3 of the steroid skeleton and are isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, the so-called mycosterols.
phospholipids. These are understood primarily as the glucose phospholipids, which are obtained e.g. as lecithins or phosphatidylcholines from e.g. egg yolk or plant seeds (e.g. soybeans).
fatty acid esters of sugars and sugar alcohols, such as sorbitol,
polyglycerols and polyglycerol derivatives, such as e.g. polyglycerol poly-12-hydroxystearate (commercial product Dehymuls® PGPH),
linear and branched fatty acids with 8 to 30 C atoms and Na, K, ammonium, Ca, Mg and Zn salts thereof.

The emulsifiers are employed preferably in quantities of 0.1-25 wt. %, in particular 0.5-15 wt. %, based on the total agent.

Suitable nonionogenic polymers are e.g.:
vinylpyrrolidone/vinyl ester copolymers, as marketed e.g. with the trademark Luviskol® (BASF). Luviskol® VA 64 and Luviskol® VA 73, both of them vinylpyrrolidone/vinyl acetate copolymers, are likewise preferred nonionic polymers.
cellulose ethers, such as hydroxypropyl cellulose, hydroxyethyl cellulose and methyl hydroxypropyl cellulose, as marketed e.g. with the trademarks Culminal® and Benecel® (AQUALON) and Natrosol® grades (Hercules).
starch and derivatives thereof, in particular starch ethers, e.g. Structure XL (National Starch), a multifunctional, salt-tolerant starch;
shellac
polyvinylpyrrolidones, as marketed e.g. with the name Luviskol® (BASF).

The nonionic polymers are contained in the compositions according to the invention preferably in quantities of 0.05 to 10 wt. %, based on the total agent. Quantities of 0.1 to 5 wt. % are more preferred.

The polymers are contained in the compositions used according to the invention preferably in quantities of 0.01 to 30 wt. %, based on the total composition. Quantities of 0.01 to 25, in particular of 0.01 to 15 wt. %, are more preferred.

The action of the compositions according to the invention can furthermore be increased by a 2-pyrrolidinone-5-carboxylic acid and derivatives thereof (J). Preferred are the sodium, potassium, calcium, magnesium or ammonium salts, in which the ammonium ion carries one to three C₁ to C₄ alkyl groups besides hydrogen. The sodium salt is particularly preferred. The quantities employed in the agents according to the invention are 0.05 to 10 wt. %, based on the total agent, more preferably 0.1 to 5 and in particular 0.1 to 3 wt. %.

Finally, further advantages are obtained through the use of plant extracts in the compositions according to the invention. According to the invention, primarily the extracts of green tea, oak bark, nettles, hamamelis, hops, henna, chamomile, burdock root, horsetail, hawthorn, lime blossom, almond, aloe vera, fir needle, horse chestnut, sandalwood, juniper, coconut, mango, apricot, lime, wheat, kiwi, melon, orange, grapefruit, sage, rosemary, birch, mallow, valerian, lady's smock, wild thyme, yarrow, thyme, melissa, rest harrow, coltsfoot, marsh mallow, meristem, ginseng, coffee, cocoa, moringa and ginger root are preferred. It can be most preferred according to the invention if so-called ayurvedic plant extracts are used as plant extracts. The traditional ayurvedic plants include Aegle marmelos (Bilva), Cyperus rotundus (Nagarmotha), Emblica officinalis (Amalaki), Morida citrifolia (Ashyuka), Tinospora cordifolia (Guduchi), Santalum album (Chandana), Crocus sativus (Kumkuma), Cinnamonum zeylanicum and Nelumbo nucifera (Kamala).

As a further essential ingredient, the agents according to the invention can contain purine and/or (a) purine derivative(s). Preferred compositions according to the invention contain purine and/or purine derivatives in relatively narrow quantitative ranges. Preferred cosmetic agents according to the invention here are characterized in that they contain—based on their weight—0.001 to 2.5 wt. %, preferably 0.0025 to 1 wt. %, more preferably 0.005 to 0.5 wt. % and in particular 0.01 to 0.1 wt. % purine(s) and/or purine derivative(s).

Among purine, the purines and the purine derivatives, the following compounds are preferred according to the invention: purine, adenine, guanine, uric acid, hypoxanthine, 6-purinethiol, 6-thioguanine, xanthine, caffeine, theobromine or theophylline. In hair cosmetic formulations, caffeine has proved particularly suitable, which can be employed e.g. in shampoos, conditioners, hair tonics and/or lotions preferably in quantities of 0.005 to 0.25 wt. %, more preferably of 0.01 to 0.1 wt. % and in particular of 0.01 to 0.05 wt. % (based in each case on the composition).

Another preferred active agent for additional use in the agents according to the invention is taurine and/or a derivative of taurine. Taurine is understood to be exclusively 2-aminoethanesulfonic acid and explicitly mentioned derivatives of taurine. The derivatives of taurine are understood to be N-monomethyltaurine and N,N-dimethyltaurine. Further taurine derivatives are also understood to be taurines which occur naturally as metabolites in vegetable and/or animal and/or marine organisms. These include for example, though not preferably, degradation products of cysteine, in particular cysteine sulfinic acid. Other taurine derivatives within the meaning of the present invention are taurocholic acid and hypotaurine.

More preferred are agents according to the invention which contain—based on their weight—0.0001 to 10.0 wt. %, preferably 0.0005 to 5.0 wt. %, more preferably 0.001 to 2.0 wt. % and in particular 0.001 to 1.0 wt. % taurine and/or a derivative of taurine.

In addition, it can prove advantageous if penetration auxiliaries and/or swelling agents (M) are contained in the compositions according to the invention. These include e.g. urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, e.g. propylene glycol monoethyl ether, carbonates, hydrogen carbonates, diols and triols, and in particular 1,2-diols and 1,3-diols such as e.g. 1,2-propanediol, 1,2-pentanediol, 1,2-hexanediol, 1,2-dodecanediol, 1,3-propanediol, 1,6-hexanediol, 1,5-pentanediol, 1,4-butanediol.

Hydantoins can be a more preferred group of swelling agents. Compositions according to the invention preferably contain 0.01 to 5 wt. % hydantoin or at least one hydantoin derivative. More preferably according to the invention, hydantoin derivatives are employed with 5-ureidohydantoin being more preferred. Regardless of whether hydantoin or hydantoin derivative(s) is/are employed, quantities of 0.02 to 2.5 wt. % are particularly preferred, of 0.05 to 1.5 wt. %, more preferably 0.075 to 1 wt. %.

Furthermore, the cosmetic agents can contain further active agents, auxiliary substances and additives, such as e.g.
structurants, such as maleic acid and lactic acid,
perfume oils, dimethyl isosorbide and cyclodextrins,
dyes for tinting the agent,
anti-dandruff active agents such as piroctone olamine, zinc omadine and climbazole,
cholesterol,
chelating agents, such as EDTA, NTA, β-alanine diacetic acid and phosphonic acids,
pigments,
antioxidants.

The single-phase hair-conditioning agents according to the invention preferably have a pH value in the acidic range of 2 to 6.5, preferably 2.4 to 6.0 and in particular 2.8 to 5.5.

With regard to further optional components and the quantities of these components employed, explicit reference is made to the relevant handbooks known to the person skilled in the art.

As already mentioned, the agents according to the invention are suitable in particular for caring for hair and they improve in particular the combability, elasticity, volume and gloss of hair.

The invention therefore thirdly provides the cosmetic use of the single-phase hair-conditioning agent according to the invention to increase hair elasticity, hair gloss and hair volume and to improve combability.

The invention fourthly provides a cosmetic method for haircare, in which the single-phase hair-conditioning agent according to the invention is sprayed onto the dry or wet hair—preferably after cleaning the hair—and left on the hair until the next cleaning of the hair.

### EXAMPLES

The following examples are intended to explain the subject matter of the present invention but without limiting it.

The quantities given relate—unless stated otherwise—to wt. %. Production took place by the method described. The steps listed in the table of the following example correspond to the production method according to the invention.

Hair Gel:

| | According to | |
|---|---|---|
| | the invention | Prior art |
| Polyquaternium-37 | 0.5 | — |
| powder | | |
| Salcare ® SC 96 | — | 0.5 |
| (based on the active | | |
| content of poly- | | |
| quaternium-37) | | |
| Quaternium-87 | 2.5 | 2.5 |

Adjustment of pH to 4.2

| | | |
|---|---|---|
| Glycerol | 20.0 | 20.0 |

Adjustment of pH to 3.5

| | | |
|---|---|---|
| Water | To 100 | To 100 |
| Appearance | Clear | Cloudy |
| Feel | Oily, rich, | Slippery, |
| | substantial | insubstantial |
| Distribution | Good to very | Uneven, greasy |
| | good, even | |
| Viscosity in mPas | 19,500 | 13,000 |

measured with a Brookfield DV-II viscometer, spindle 4 at 20 rpm (20 s) and at 20° C.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A transparent hair-conditioning agent in gel form, containing
a) a cationic polymer, consisting of at least one monomer of formula (I) and optionally a monomer of formula (II) 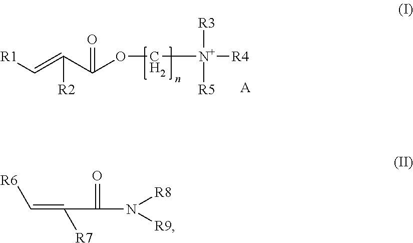
in which R1 to R9 independently of one another signify hydrogen, C₁₋₄ alkyl, methyl, ethyl, propyl, isopropyl, butyl, isobutyl or tert-butyl with the proviso that at least one of the residues R6, R7, R8 or R9 signifies C₁₋₄ alkyl, n denotes integers from 1 to 8 and A denotes a physiologically acceptable anion such as halide, fluoride, chloride, bromide, iodide, hydrogen sulfate or metho sulfate,
b) at least one cationic compound of formula (Tkat2), 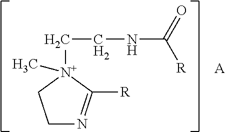
in which the residues R each independently of one another denote a saturated or unsaturated, linear or branched hydrocarbon residue with a chain length of 8 to 30 carbon atoms and A denotes a physiologically acceptable organic or inorganic anion and is selected from the halide ions, fluoride, chloride, bromide, iodide, sulfates of the general formula RSO₃⁻, where R has the meaning of saturated or unsaturated alkyl residues with 1 to 4 carbon atoms, or anionic residues of organic acids such as maleate, fumarate, oxalate, tartrate, citrate, lactate or acetate, and
c) 15.0 wt % to 55.0 wt. % of at least one polyhydroxy compound selected from the group consisting of glycol, glycerol, 1,2-propylene glycol, 1,3-propylene glycol, 1,2-pentanediol, 1,5-pentanediol, 1,2-hexanediol, 1,6-hexanediol, and mixtures thereof; and
d) a cosmetic carrier.

2. A method of producing a cosmetic composition according to claim 1, characterized by the following steps:
a) pre-swelling of the cationic polymer according to claim 1 at a temperature of 15 to 50° C.,
b) addition of the cationic surfactant according to claim 1 with further stirring,
c) adjustment of the pH value to 4 to 5 and optional addition of further water-soluble substances,
d) addition of the polyhydroxy compound according to claim 1 with stirring and optional final adjustment of the pH value of the composition to a pH value of 2 to 6.5.

3. The hair-conditioning agent according to claim 1, wherein the hair-conditioning agent has a viscosity of 15000 to 50000 mPas (measured with a Brookfield DV-II viscometer, spindle 4 at 20 rpm (20 s) and at 20° C.).

4. The hair-conditioning agent according to claim 1, wherein the hair-conditioning agent has a transparency of at least 50 to 100%, measured in accordance with DIN/EN 27027.

5. The hair-conditioning agent according to claim 1, wherein the polyhydroxy compound is selected from the group consisting of: glycol, glycerol, 1,2-propylene glycol, 1,3-propylene glycol, and mixtures thereof.

6. The hair-conditioning agent according to claim 1, further comprising at least one oil, wax and/or fat component in an amount of 0.05 wt % to 15 wt % based on the total weight of the hair-conditioning agent.

7. The hair-conditioning agent according to claim 6, wherein the oil, wax and/or fat component includes at least one silicone and/or at least one vegetable oil.

8. The hair-conditioning agent according to claim 1, further comprising at least one substance selected from the group consisting of the vitamins, provitamins, vitamin precursors, and substance from the group of the protein hydrolyzates.
